(19) United States
(12) Patent Application Publication
(10) Pub. No.: US 2001/0047139 A1
(43) Pub. Date: Nov. 29, 2001

(54) Method for diagnosing the spine

(76) Inventor: David R. Toftness, Amery, WI (US)

Correspondence Address: Tipton L. Randall, 19371 55th Avenue, Chippewa Falls, WI, 54729, US

(21) Appl. No.: 09/781,489

(22) Filed: Feb. 13, 2001

Related U.S. Application Data
(60) Provisional application No. 60/183,292, filed on Feb. 17, 2000.

(51) Int. Cl. A61B 5/103

(52) U.S. Cl. 600/594; 600/587; 600/409

(57) ABSTRACT

A method of diagnosing the spine of a patient is provided, the method comprising (a) placing a patient to be diagnosed in a prone position; (b) providing a magnetometer; (c) identifying a vertebra of the spine to be diagnosed; (d) placing the magnetometer over the vertebra to be diagnosed and obtaining a magnetometer reading of the vertebra; (e) identifying at least one additional vertebra to be diagnosed and repeating step (d) for the at least one additional vertebra, and (f) interpreting the data to identify a subluxated vertebra. The method also includes delivering a chiropractic adjustment to the subluxated vertebra identified. A subluxated vertebra is thus identified using a magnetometer.

### CROSS REFERENCE TO RELATED APPLICATION

This application claims the benefit under 35 U.S.C. §119(e) of co-pending provisional application Serial No. 60/183,292, filed Feb. 17, 2000. application Ser. No. 60/183,292 is hereby incorporated by reference.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### FIELD OF THE INVENTION

The present invention relates to a method of diagnosing and treating the spine of a patient, and more particularly, to a method of diagnosing and treating the spine of a patient employing a magnetometer.

### BACKGROUND OF THE INVENTION

Over 100 years ago, it was discovered that vertebrae in the spine that are displaced slightly (chiropractic subluxation) can cause neck pain, low back pain, headaches and a whole host of internal conditions including, but not limited to, dysmenorrhea, colitis, and otitis media. Since the discovery of subluxation, practitioners have been trying to find better ways to locate and correct spinal subluxations. To date, many methods have been used. These include palpation of the spine to feel physical displacement, x-ray of the spine to observe the physical displacement, and infrared and millimeter wave thermography of the spine to locate the displacement by the interference that it causes on the nervous system.

The methods used to date to locate spinal subluxations have disadvantages. For example, palpation of the spine requires that the practitioner be highly trained, and the inter-examiner reliability has been shown to be only moderately reliable. X-rays have a certain amount of danger associated with them and the image is only a snapshot in time. Infrared and millimeter wave thermography are associated with instrument errors and environmental errors that makes the use of them less than perfect. It would be beneficial to humanity if these disadvantages could be overcome.

Currently, there is a need of a method for diagnosing the spine that is repeatable, sensitive and non-invasive, with an instrument that is portable and provides immediate information regarding the status of the spine. There is a need of a method for diagnosing the spine that does not carry with it the disadvantages noted herein above.

### SUMMARY OF THE INVENTION

The present invention provides a method for diagnosing the spine that is repeatable, sensitive and non-invasive. The present invention provides a method for diagnosing the spine with an instrument that is portable and provides immediate information regarding the status of the spine. The present invention provides a method for characterizing and diagnosing spinal health using an electromagnetic or magnetic device. The method of the present invention is contemplated as being applicable to body areas other than the spine, as well.

A method of diagnosing a body area, such as the spine, of a patient is provided. The method comprises; (a) placing a patient to be diagnosed in a prone position; (b) providing a magnetometer; (c) identifying an initial body area, such as a vertebra of the spine, to be diagnosed; (d) placing the magnetometer over the initial body area, such as a vertebra, to be diagnosed and taking a first magnetometer reading of the initial body area, such as a vertebra; and (e) determining whether the initial body area, such as a vertebra, is normal or is abnormal by interpreting data of the first magnetometer reading.

Where the initial body area is a vertebra of the spine, and the initial vertebra is determined to be normal or without interference, the method further includes; (f) identifying at least one additional vertebra to be diagnosed and repeating steps (d) and (e) for the at least one additional vertebra; and (g) interpreting the data to identify an abnormal or subluxated vertebra. The method also includes delivering a chiropractic adjustment to the abnormal or subluxated vertebra. A subluxated vertebra is thus identified using a magnetometer.

### BRIEF DESCRIPTION OF THE DRAWINGS

FIG. 1 is a conceptual side view of a human spine.

FIG. 2 is a flow chart of one method of the present invention.

FIG. 3 is a flow chart of another method of the present invention.

FIG. 4 is a flow chart of another method of the present invention.

FIG. 5 is a flow chart of another method of the present invention.

While the invention is amenable to various modifications and alternative forms, specifics thereof have been shown by way of example in the drawings and will be described in detail. It should be understood, however, that the intention is not necessarily to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

The present invention is believed to be applicable to a variety of different human body areas, and has been found to be particularly suited for diagnosing abnormal or subluxated vertebra of the spine, as well as determining when treatment has restored the vertebra to a normal condition, that is, without interference. While the present invention is not necessarily limited to diagnosis and treatment of the vertebra of the spine, various aspects of the invention may be appreciated through a discussion of various examples using this context.

According to an example embodiment of the present invention, a method of diagnosing a body area, such as the spine, of a patient is provided. The method includes placing a patient to be diagnosed in a prone position. A magnetometer is provided, and an initial body area, such as a vertebra of the spine, to be diagnosed is identified. The magnetometer is placed over the initial body area, such as a vertebra, to be diagnosed and a first magnetometer reading is taken of the initial body area, such as a vertebra. Interpreting data of the first magnetometer reading determines whether the initial body area, such as a vertebra, is normal or is abnormal.

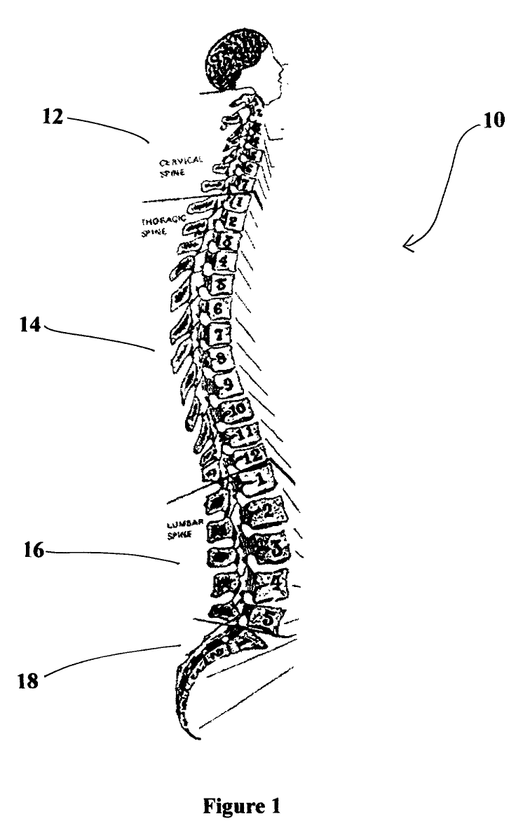

Referring to FIG. 1, a human spine is shown. The human spine 10 has twenty six vertebrae, which are divided into four regions. These regions are the cervical region 12 which has seven vertebrae, the thoracic region 14 which has twelve vertebrae, the lumbar region 16 which has five vertebrae and the sacrum region 18 which has two vertebrae.

When spinal vertebra in a human are out of alignment, the nervous system is disturbed and symptoms and/or disease ensue. When vertebra are replaced to their normal juxtaposition, the nervous system interference is reduced and health returns. The nervous system is electrical in nature and exhibits known electrical properties including, but not limited to, conduction, EMF fields and the like.

The earth has a magnetic field of its own. Extensive mapping of this magnetic field has been achieved employing magnetometers. The magnetic field is commonly measured along the X, Y and Z axis of the Cartesian coordinate system. Each of the three components (X, Y, Z) has a different magnitude, and the magnitude of each component varies from location to location. The vertical magnetic component (Z axis) varies in the range of about 36,000 to about 54,000 nanoTesla (nT) or about 360 to about 540 milligauss (mG) within the United States.

The method of the present invention is based on the Applicant's discovery that the human spine emits a magnetic field that is superimposed upon the earth's magnetic field, and that abnormal or subluxated spinal vertebrae superimpose a magnetic field of greater magnitude than do normal (without interference) vertebrae. By measuring the magnetic field vertical or Z axis component of the spine of a patient in the prone position, the condition of the spinal vertebrae can be diagnosed. It has been discovered that the human spine that provides a magnetic field vertical or Z axis component value of less than about 420 mG is considered to be baseline and without interference. The spine that has a magnetic field vertical or Z axis component value of more than about 420 mG is found to be associated with a disturbed nervous system (subluxation).

One instrument that has been discovered as being capable of use for obtaining magnetic measurements of the spine is the Walker Scientific Fluxgate Magnetometer. The method of the present invention, however, is intended to encompass the use of any instrument which measures electromagnetic or magnetic force or any magnetometer. Although the present invention is not intended to be limited thereby, information regarding Walker Scientific's Fluxgate Magnetometers is available from Walker Scientific, located on Rockdale Street, Worcester, Mass., 01606. Examples of Walker Scientific Fluxgate Magnetometers which may be utilized in the method of the present invention are the FGM-4DTAM Triaxial Magnetometer, the FGM-5DTAA Triaxial Magnetometer, the FGM-3D2L Single Axis Fluxgate Magnetometer, the FGM-3D2T Single Axis Fluxgate Magnetometer, the FGM-4D2L Single Axis Fluxgate Magnetometer, the FGM-4D2T Single Axis Fluxgate Magnetometer, the FGM-3D2LN Single Axis Fluxgate Magnetometer, the FGM-3D21N Single Axis Fluxgate Magnetometer, the FGM-4D2LN Single Axis Fluxgate Magnetometer and the FGM-4D2TN Single Axis Fluxgate Magnetometer. The measurement is taken with the Walker Scientific Fluxgate Magnetometer from above the patient, using the vertical or Z axis of the magnetometer probe. The single axis probe is positioned above the prone patient's spine with the single probe axis oriented vertically downward. Alternatively, the triaxial probe is also positioned above the prone patient's spine with the triaxial probe Z axis oriented vertically downward.

In one embodiment of the method of the present invention, the patient is placed in a prone position. The Fluxgate magnetometer probe is placed successively over each vertebra of the spine that the practitioner selects for diagnosis, and a magnetometer reading value is obtained for each. The vertebra of the spine that has the highest reading value, greater than about 420 mG, is diagnosed as an abnormal or subluxated vertebra. A chiropractic adjustment is delivered to the abnormal or subluxated vertebra that has the highest reading. The chiropractic adjustment may be made according to any chiropractic adjustment system. For example, an adjustment may be made using the Toftness System, described in the articles “Evaluation of the Toftness System of Chiropractic Adjusting for the Relief of Acute Pain of Musculoskeletal Origin” and “Evaluation of the Toftness System of Chiropractic Adjusting for Subjects with Chronic Back Pain, or Chronic Tension Headaches or Primary Dysmenorrhea”, published in Chiropractic Technique in May, 1992, and February, 1996, respectively.

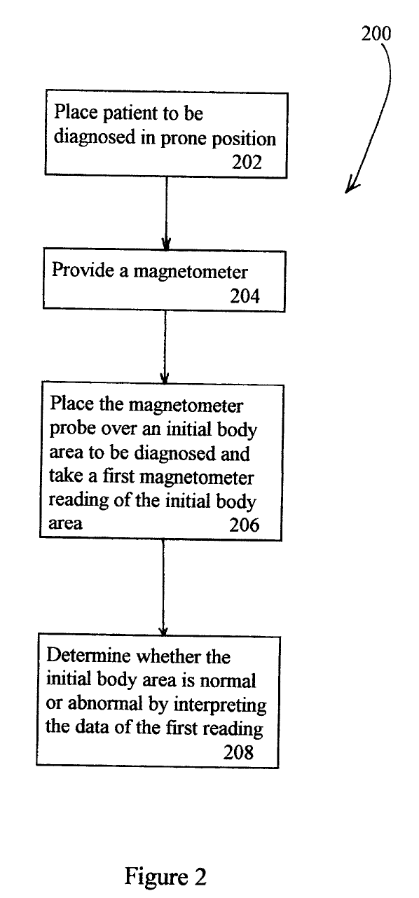

A magnetometer reading is again taken of the vertebra or vertebral area following the chiropractic adjustment. A chiropractic adjustment to the spine is complete and successful when the reading value over the vertebra returns to the baseline, that is less than about 420 mG. This method may alternatively be performed on the vertebral regions of the spine. For example, one magnetometer reading value would be taken of the cervical area, a chiropractic adjustment would be made to the cervical area and a second magnetometer reading value would be taken of the cervical area following the chiropractic adjustment. This process would be repeated for each of the remaining three vertebral regions. FIGS. 2-5 are flow chart diagrams of the several embodiments of the method of the present invention.

Referring to FIG. 2, the method 200 is shown. A patient to be diagnosed is placed in a prone position (202). A magnetometer is provided (204). The magnetometer probe is placed over an initial body area to be diagnosed and a first magnetometer reading of the initial body area is taken (206). It is determined by interpreting the data of the first reading whether the initial body area is normal or abnormal (208). The determination whether the initial body area is normal or abnormal is made by comparing the first magnetometer reading value with a baseline value established for the particular body area that is deemed normal.

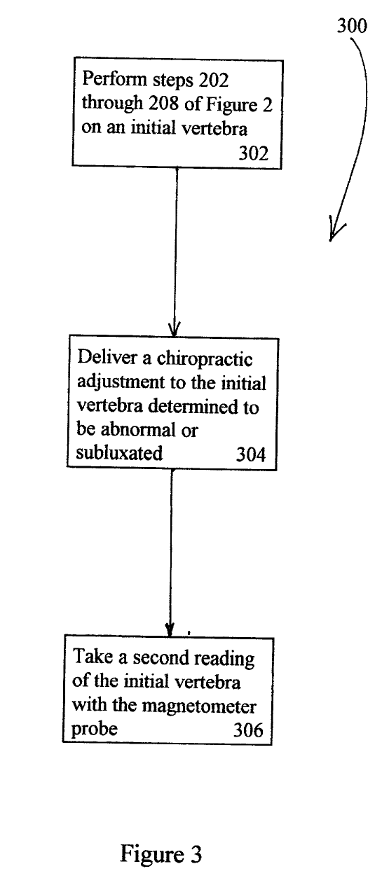

Referring to FIG. 3, method 300 is shown. Steps 202-208 of FIG. 2 are repeated where the initial body area is an initial vertebra of the spine (302). The determination whether the initial vertebra is normal or abnormal is made by comparing the first magnetometer reading value with a baseline value established for the vertebra that is deemed normal. The baseline value for normal vertebra is about 420 mG. In the method of FIG. 3, it is assumed that the initial vertebra is determined to be abnormal or subluxated. A chiropractic adjustment is delivered to the initial vertebra determined to be subluxated (304). A second magnetometer reading is taken of the initial vertebra with the magnetometer (306) to determine whether the initial vertebra is now normal, that is, without interference. A magnetometer reading value of less than about 420 mG indicates a normal vertebra.

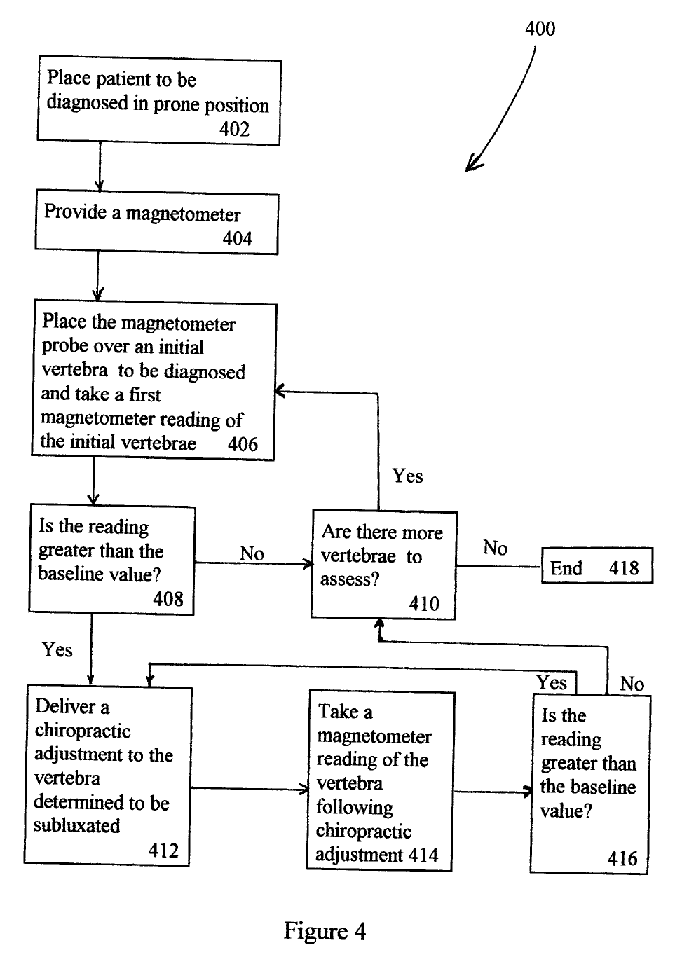

Referring now to FIG. 4, method 400 is shown. The patient to be diagnosed is placed in a prone position (402). A magnetometer, such as a Walker Scientific Fluxgate Magnetometer, is provided (404). The magnetometer probe is placed over a vertebra to be diagnosed and a first magnetometer reading is taken (406). If the reading value is not greater than an established baseline value (about 420 mG) for vertebra (408), the process is repeated for additional vertebrae until there are no more vertebrae to assess (410). If the reading for any vertebra is greater than an established baseline value for vertebra (about 420 mG), a chiropractic adjustment is delivered to the vertebra determined to be subluxated (having a value greater than about 420 mG) (412). A magnetometer reading is taken (414) following the adjustment. If the reading value is below an established baseline value for vertebra (about 420 mG), the process is complete for that vertebra (416). If not, a further chiropractic adjustment to the vertebra may be made and a subsequent magnetometer reading taken until the reading value is below the baseline value (about 420 mG). The process continues until there are no more vertebrae to assess and treat (418).

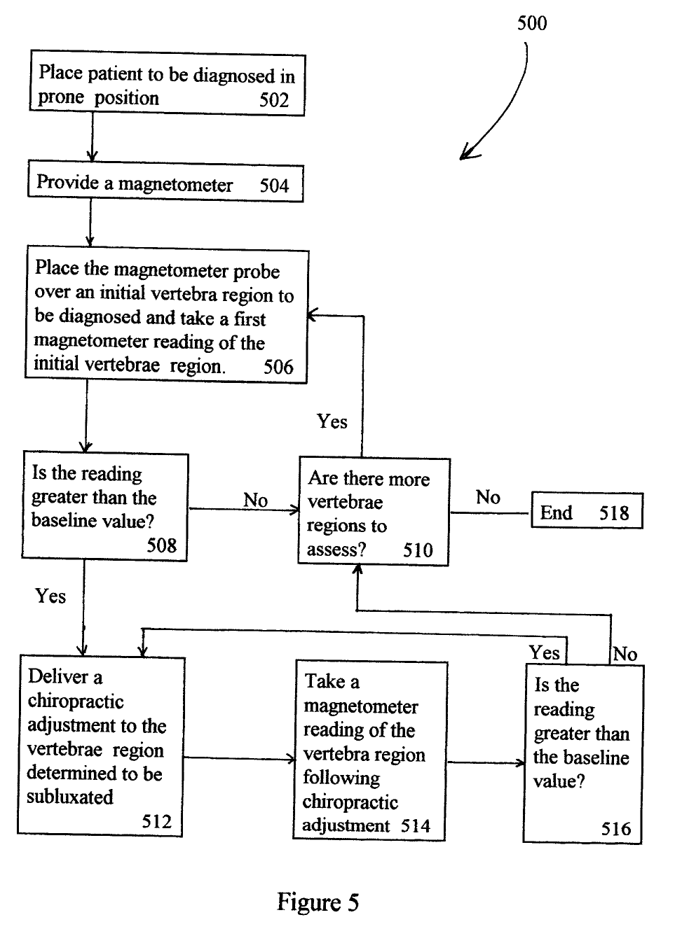

The methods set forth in FIGS. 2-4 are suitable for assessment and treatment of body areas such as spinal regions. Referring now to FIG. 5, following the steps (502) and (504), the initial magnetometer reading (506) is performed on a vertebral region instead of a single vertebra. If the vertebral region produces a magnetometer reading value above an established baseline value (about 420 mG) (508), a chiropractic adjustment to the vertebral region is made (512). A follow up magnetometer reading is taken (514) to determine if the reading value is below the established baseline value (about 420 mG) for the vertebral region (516). This process is then repeated for each of the three remaining vertebral regions (510).

### EXAMPLE

Method: Forty-four randomly selected subjects were assigned into control (20 subjects) and experimental groups (24 subjects) in a pre- and post-test design. Subjects in the control group received no chiropractic adjustment. A Triaxial Fluxgate Magnetometer FGM-5DTAA (Walker Scientific, Worcester, Mass.) with five digit display and resolution of 1 nT in a 100,000 nT field was used for magnetic field detection. Thus, small variations in magnetic field can be measured in the presence of a large field, such as the earth's magnetic field. The magnetic field components can be displayed in nanotesla (nT), microtesla (mT) or milligauss (mG). The FGM-5DTAA instrument has a sample rate of 69 samples per second for real time magnetic field measurement. The instrument was calibrated according to the industrial standards for accurate magnetic field readings.

The magnetic field in the research room and on the adjustment table was monitored and recorded. The room magnetic field was measured in three dimensions, namely the X, Y and Z axes. While holding the instrument probe horizontally during testing, the X axis is detecting the magnetic field component from the south, the Y axis is detecting magnetic field component from the east, and the Z axis is detecting the magnetic component field from the ground (earth field). The room magnetic field was 416.11 mG at the Z axis, 137.61 mG at the X axis and 74.38 mG at the Y axis. The plain wood table had magnetic field readings in the range of 410.00 mG at the Z axis. A motorized Hi-Lo table had much higher magnetic field readings of 730.00 mG at the Z axis near the lumbar and sacral regions. Only the plain wood table was used in the study for magnetic measurements and for chiropractic adjustment. The magnetic field on the table with and without the subject and at different times was recorded. The magnetic field components did not change significantly over time with the subjects lying on this table.

The subjects' body surface (cervical, thoracic, lumbar and sacral areas) magnetic field Z axis component was determined in the prone position before and after the chiropractic adjustment. Magnetic field strength along the Z axis at the cervical, thoracic, lumbar and sacral areas were measured before the chiropractic adjustment at 5, 10 and 15 minutes of lying on the table. The average Z axis magnetic field reading from the 24 subjects in the experimental group was 424.61 mG at the cervical area, 423.83 mG at the thoracic area, 433.16 mG at the lumbar area, and 434.40 mG at the sacral area prior to chiropractic adjustment. A low force Toftness chiropractic adjustment was applied to the cervical, thoracic, lumbar and sacral areas as determined by the practitioner.

Results: The 24 subjects in the experimental group showed a significant decrease in Z axis magnetic field (mean±SD in mG) after Toftness chiropractic adjustment at the cervical region from 424.49±9.07 mG to 416.43±11.65 mG (p<0.01) and at the sacral regions from 432.06±7.60 mG to 427.13±5.52 mG (p<0.01). The Z axis magnetic field at the lumbar region decreased from 429.73±7.67 mG to 428.22±9.39 (p>0.05) and the Z axis magnetic field decreased at the thoracic region from 424.59±7.44 mG to 422.25±9.81 mG (p>0.05), but did not reach a statistically significant level for these two spinal regions. The average decrease of the Z axis magnetic field in the cervical region and in the sacral region was 8.06 mG and 4.93 mG.

In the control group, Z axis magnetic field readings were taken in the same fashion as in the experimental group but there were no significant Z axis magnetic field changes in all four spinal locations.

Referring to TABLE 1, data taken in the example using the method disclosed herein are presented where n equals the number of subjects (persons) observed. According to the method, a chiropractic adjustment to the spine is complete and successful when the magnetometer reading value over the vertebra returns to the baseline, which is about 420 mG. The p value of less than 0.05 is statistically significant and is another indicia of successful treatment.

TABLE 1
Magnetometer Measurements Before and After Chiropractic Adjustment

| | n | MEAN (mG) | SD (mG) | P |
|---|---|---|---|---|
| Cervical (before) | 24 | 424.49 | 9.07 | 0.00091 |
| Cervical (after) | 24 | 416.43 | 11.65 | |
| Thoracic (before) | 24 | 424.59 | 7.44 | 0.113 |
| Thoracic (after) | 24 | 422.25 | 9.81 | |
| Lumbar (before) | 24 | 429.73 | 7.67 | 0.348 |
| Lumbar (after) | 24 | 428.22 | 9.39 | |
| Sacrum (before) | 24 | 432.06 | 7.60 | 0.00445 |
| Sacrum (after) | 24 | 427.13 | 5.52 | |

The method of the present invention eliminates the dangers and disadvantages of prior methods of locating spinal subluxations. The method eliminates the unreliability of palpation as a diagnostic tool, the danger of x-rays, the necessity for a magnetically shielded room, and instrumental and environmental error associated with infrared and millimeter wave thermography. The use of a magnetometer and the Fluxgate Magnetometer in particular provides a method of diagnosis and treatment of spinal subluxation that is repeatable, reliable, sensitive, non-invasive, and portable, while providing immediate information on the status of the spine.

The invention has been described with reference to various specific and preferred embodiments and techniques. However, it should be understood that many variations and modifications may be made while remaining within the spirit and scope of the invention.

What is claimed is:

1. A method of diagnosing and treating a patient, the method comprising:
(a) placing a patient to be diagnosed in a prone position;
(b) providing a magnetometer;
(c) identifying an initial body area of the patient to be diagnosed;
(d) placing the magnetometer over the initial body area to be diagnosed and taking a first magnetometer reading of the initial body area, and
(e) determining whether the initial body area is normal or is abnormal by interpreting data of the first magnetometer reading.

2. The method of claim 1 wherein determining whether the initial body area is normal or abnormal by interpreting data of the first magnetometer reading includes establishing a magnetometer reading baseline value for a normal initial body area, and comparing the first magnetometer reading value of the initial body area of the patient with the magnetometer reading baseline value for a normal initial body area.

3. The method of claim 2 wherein identifying an initial body area and taking a first magnetometer reading of the initial body area of the patient includes identifying an initial vertebra and taking a first magnetometer reading of an initial vertebra of the spine of the patient.

4. The method of claim 1 wherein providing a magnetometer includes providing a Fluxgate magnetometer and taking a first magnetometer reading includes taking a magnetometer reading on the Z axis over the initial body area.

5. The method of claim 3 wherein a magnetometer reading baseline value of about 420 mG is established for a normal vertebra of the spine, and a subluxated vertebra of the spine is identified by determining whether the initial vertebra has a magnetometer reading value greater than about 420 mG.

6. The method of claim 3 wherein a magnetometer reading baseline value of about 420 mG is established for a normal vertebra of the spine, and a vertebra of the spine without interference is identified by determining whether the initial vertebra has a magnetometer reading of less than about 420 mG.

7. The method of claim 3 further comprising;
(f) delivering a chiropractic adjustment to an initial vertebra determined to be abnormal, and
(g) taking a second magnetometer reading of the initial vertebra with the magnetometer to determine if the initial vertebra gives a magnetometer reading value less than the baseline value.

8. A method of diagnosing and treating a spine of a patient, the method comprising:
(a) placing a patient to be diagnosed in a prone position;
(b) providing a magnetometer;
(c) identifying an initial vertebra of the spine to be diagnosed;
(d) placing the magnetometer over the initial vertebra to be diagnosed and taking a first magnetometer reading of the initial vertebra, and
(e) determining whether the initial vertebra is without interference or is subluxated by interpreting data of the first magnetometer reading.

9. The method of claim 8 wherein providing a magnetometer includes providing a Fluxgate magnetometer and taking a first magnetometer reading includes taking a magnetometer reading on the Z axis over the initial vertebra.

10. The method of claim 8 wherein determining whether the initial vertebra is without interference or is subluxated by interpreting data of the first magnetometer reading includes establishing a magnetometer reading baseline value for a normal vertebra, and comparing the first magnetometer reading value of the initial vertebra of the patient with the magnetometer reading baseline value for a normal initial vertebra.

11. The method of claim 10 wherein a magnetometer reading baseline value of about 420 mG is established for a normal vertebra, and a subluxated vertebra is identified by determining whether the initial vertebra has a magnetometer reading value greater than about 420 mG.

12. The method of claim 10 wherein a magnetometer reading baseline value of about 420 mG is established for a normal vertebra, and a vertebra without interference is identified by determining whether the initial vertebra has a magnetometer reading value of less than about 420 mG.

13. The method of claim 8 further comprising;
(f) delivering a chiropractic adjustment to an initial vertebra determined to be subluxated, and
(g) taking a second magnetometer reading of the initial vertebra with the magnetometer to determine if the initial vertebra gives a magnetometer reading value less than the baseline value.

14. The method of claim 13 further comprising;
(h) upon determining the second magnetometer reading value to be greater than the baseline value, repeating steps (f) and (g) until the magnetometer reading value is less than the baseline value.

15. A method of diagnosing and treating a spine of a patient, the method comprising:
(a) placing a patient to be diagnosed in a prone position,
(b) providing a magnetometer,
(c) diagnosing a subluxated vertebra by
(i) identifying an initial vertebra of the spine to be diagnosed,
(ii) placing the magnetometer over the initial vertebra to be diagnosed and taking a first magnetometer reading of the vertebra,
(iii) determining whether the initial vertebra is subluxated or is without interference by interpreting data from the first magnetometer reading taken,
(iv) upon determining the initial vertebra is without interference, identifying at least one additional vertebra to be diagnosed and repeating steps (i)-(iii) until identifying a subluxated vertebra,
(d) delivering a chiropractic adjustment to the subluxated vertebra diagnosed in (c), and
(e) taking a second magnetometer reading of the subluxated vertebra diagnosed in (c) following the chiropractic adjustment to confirm that the identified vertebra is without interference.

16. The method of claim 15 wherein providing a magnetometer includes providing a Fluxgate magnetometer and taking a first magnetometer reading includes taking a magnetometer reading on the Z axis over the initial vertebra.

17. The method of claim 15 wherein determining whether the initial vertebra is without interference or is subluxated by interpreting data of the first magnetometer reading includes establishing a magnetometer reading baseline value for a normal vertebra, and comparing the first magnetometer reading value of the initial vertebra of the patient with the magnetometer reading baseline value for a normal initial vertebra.

18. The method of claim 15 wherein a magnetometer reading baseline value of about 420 mG is established for a normal vertebra, and a subluxated vertebra is identified by determining whether the initial vertebra has a magnetometer reading value greater than about 420 mG.

19. The method of claim 15 wherein a magnetometer reading baseline value of about 420 mG is established for a normal vertebra, and a vertebra without interference is identified by determining whether the initial vertebra has a magnetometer reading value of less than about 420 mG.
